(19) United States
(12) Patent Application Publication
(10) Pub. No.: US 2023/0340562 A1
(43) Pub. Date: Oct. 26, 2023

(54) MOISTURE INDUCED BIOLUMINESCENT COMPOSITION AND METHODS FOR MANUFACTURING THEREOF

(71) Applicant: LULULEMON ATHLETICA CANADA INC., Vancouver (CA)

(72) Inventors: Felix MOSER, Vancouver (CA); Lynn Yuqin Wan, Vancouver (CA); Timothy Ryan McGee, Vancouver (CA)

(21) Appl. No.: 18/300,194

(22) Filed: Apr. 13, 2023

Related U.S. Application Data
(60) Provisional application No. 63/331,061, filed on Apr. 14, 2022.

(51) Int. Cl. C12Q 1/66

(52) U.S. Cl. CPC C12Q 1/66

(57) ABSTRACT

Examples of a moisture induced bioluminescent composition are disclosed. The moisture induced bioluminescent composition includes luciferin granulates and a mixture of a dried luciferase enzyme and one or both of at least one cofactor and at least one excipient. The luciferin granulates are mixed with the dried luciferase mixture forming an unreactive composition that is configured to produce a luminescence when exposed to moisture.

### TECHNICAL FIELD

The present disclosure relates to the field of bioluminescent compositions, and more specifically, to a composition comprising bioluminescent reagents that are activated when in contact with moisture.

### BACKGROUND

Luciferases are enzymes that oxidize luciferins, small molecules that generate photons upon oxidation. This reaction is used throughout nature for bioluminescence and plays various ecological roles. Both luciferases and luciferins have a wide range of structures, biochemistries, stabilities, activity optima, and colors of emitted light (as described in Kotlobay, Kaskova, & Yampolksy (2020). Acta Naturae). These bioluminescent systems have found broad applicability throughout biotechnology, serving primarily as reporters of gene expression in multiple organisms (Fleiss & Sarkisyan (2019). Current Genetics). Furthermore, bioengineers have altered the properties of various luciferins to increase their brightness, the color of the emitted photons, specificities, and stabilities (as described in Kim, et al. (2011). Analytical Chemistry; Gregor, et al. (2017). PNAS; and Pozzo, et al. (2018). ACS Omega).

In the past decade, freeze-drying has been tested as a means of preserving enzymes and other biomolecules for long periods of time prior to use (Pardee, et al (2014). Cell).

However, there remains a need for bioluminescent compositions that can be added to a variety of surfaces and substrates and that are activated by the presence of moisture.

### SUMMARY

In one aspect, a moisture induced bioluminescent composition is provided. The moisture induced bioluminescent composition includes luciferin granulates, and a mixture of a dried luciferase enzyme(s) and one or both of at least one cofactor and at least one excipient. For example, the mixture may comprise the dried luciferase enzyme(s), the at least one cofactor and the at least one excipient. Alternatively, the mixture may comprise the dried luciferase enzyme(s) and the at least one cofactor, or the mixture may comprise the dried luciferase enzyme(s) and the at least one excipient. In various embodiments, the luciferin granulates are mixed with the dried luciferase mixture forming an unreactive composition that is configured to produce luminescence when exposed to moisture.

In various embodiments, the luciferin granulates comprise a D-luciferin, a coelenterazine, and a vargulin/cypridinaluciferin.

In various embodiments, the luciferase enzyme is purified or partially purified from a microbial culture.

In various embodiments, the at least one excipient is selected from the group consisting of a sucrose, a trehalose, a mannitol, a polyvinylpyrrolidone (PVP), a polysorbate, a dextrose, a glycine and a combination thereof.

In various embodiments, the at least one cofactor is a flavin cofactor, a flavin mononucleotide (FMN) and/or a nicotinamide adenine dinucleotide.

In various embodiments, the moisture induced bioluminescent composition further comprises at least one of an adhesive, a surfactant, a thickener, or a dispersant. For example, the at least one adhesive may be selected from the group consisting of a polyurethane, a polyimide, an epoxy, an acrylic, a polyamide, a polyester and a protein.

In another aspect, a method for manufacturing a moisture induced bioluminescent composition is provided. The method comprises purifying a dissolved luciferase from a microbial fermentation culture to form a luciferase solution; mixing the luciferase solution with at least one cofactor and/or at least one excipient forming a luciferase mixture; drying the luciferase mixture using a lyophilization technique or a spray-drying technique to form a dried luciferase mixture; mixing the dried luciferase mixture with a dried luciferin; grinding the mixture of dried luciferase mixture and the dried luciferin to form a dried unreactive bioluminescent composition and packaging the unreactive bioluminescent composition to form the moisture induced bioluminescent composition.

In various embodiments, the method further comprises mixing at least one of an adhesive, a surfactant, a thickener, or a dispersant with the dried unreactive bioluminescent composition.

In various embodiments, the lyophilization technique comprises freezing the luciferase mixture at a temperature of 0° C. to −80° C. at a predetermined rate of freezing to form a frozen luciferase mixture, primary drying the frozen luciferase mixture at a pressure of 750 Torr to 40 mTorr for 8 to 12 hours and maintaining the temperature of 0° C. to −80° C. to sublimate a frozen liquid away from the frozen luciferase mixture; and secondary drying the frozen luciferase mixture at temperature of 1 to 30° C. while maintaining the pressure of 750 Torr to 40 mTorr to remove any remaining liquid from the luciferase mixture to form the dried luciferase mixture.

In various embodiments, the spray technique comprises dispersing the luciferase mixture through a spray nozzle into a hot drying gas, wherein a flow rate of the luciferase mixture is 1 to 50 ml/min, a flow rate of the hot gas is 100- to 1600 l/h, a temperature is −20° C. to 100° C., and a pressure is 750 Torr to 40 mTorr.

In various embodiments, the unreactive bioluminescent composition is packaged as an aerosol or a paste. In alternative embodiments, the unreactive bioluminescent composition is packaged in a capsule.

In a further aspect, an article of apparel is provided. The article of apparel comprises a body configured to fit and cover at least a portion of a body of a wearer, the body having an outer surface facing away from the body of the wearer, and a moisture induced bioluminescent composition as described herein applied to at least a part of the outer surface of the body.

In various embodiments, the moisture induced bioluminescent composition is coated, sprayed, printed, painted or brushed to the part of the outer surface of the body.

In addition to the aspects and embodiments described above, further aspects and embodiments will become apparent by reference to the drawings and study of the following detailed description.

### BRIEF DESCRIPTION OF THE DRAWINGS

FIG. 1 shows a scheme of an example of a method of manufacturing a moisture induced bioluminescent composition, in accordance with an embodiment of the invention.

FIG. 2 shows an example of an article of apparel with a moisture induced bioluminescent composition applied thereon, in accordance with an embodiment of the invention.

FIG. 3 shows an example of an exercise mat with a moisture induced bioluminescent composition applied on its surface, in accordance with an embodiment of the invention.

### DETAILED DESCRIPTION OF SPECIFIC EMBODIMENTS

In the context of the present disclosure, various terms are used in accordance with what is understood to be the ordinary meaning of those terms.

The term “bioluminescence” refers to production and emission of light by a living organism. It is a form of a chemiluminescence where light is released by a chemical reaction. Generally, the chemical reaction in bioluminescence involves a light-emitting molecule luciferin and an enzyme luciferase that catalyzes oxidation of the luciferin molecule. The luciferin reacts with oxygen to produce oxyluciferin and light energy.

The invention described herein discloses a bioluminescence composition activated when exposed to moisture and a method for generating the bioluminescence composition. The bioluminescent composition is a dried unreactive formulation of luciferase enzyme and respective reagents (such as luciferin and other oxidizing cofactors) that luminesces upon rehydration with water, such as with sweat. This formulation can be sprayed onto articles of clothing to enable the wearer to experience induced luminescence from their own sweat. The formulation can also be mixed with chemicals that improve adhesion to target materials, including diverse textiles, sports equipment or garments, skin, or hair. The formulation can also be incorporated or encapsulated in fibers, inks, coatings or paints for further development of end products such as textiles and accessories, sport equipment, artifacts and ornaments. The formulation can be applied by spraying, brushing, painting, printing (such as, for example, 3D printing), or by means of a secondary implement, such as a laundry capsule. In the dried formulation, the enzyme is unreactive and does not produce light. When wetted by water resources such as sweat, rain, and moisture, the enzyme is hydrated and begins to react with its accompanying reagents, producing a distinct glow that is visible in the dark.

Luciferase enzymes are naturally occurring enzymes that occur in fireflies (Photinus pyralis, Luciola cruciate, Luciola italic, Luciola lateralis, Luciola mingrelica, Photuris pennsylvanica), click beetles (Pyrophorus plagiophthalamus), railroad worms (Phrixothrix hirtus), or sea creatures (Renilla reniformis, Gaussia princeps, Cypridina noctiluca, Cypridina hilgendorfii, Metridia longa, Oplophorus gracilorostris). The luciferase enzymes catalyze the oxidation of luciferin molecules. The luciferin reacts with oxygen, producing oxyluciferin and emitting light energy (releases photons upon oxidation). The luciferin can be one or more of common luciferins that include a D-luciferin, a coelenterazine, and a vargulin/cypridinaluciferin.

Variants of both luciferase and luciferin molecules may be used to achieve different kinetic, luminescent, or chemical properties. Furthermore, the enzymes could be a new, engineered variant that has been purposefully modified to remain stable in optimal conditions, to emit different colors of light, to use different substrate luciferins, to adhere to selected materials, or to respond as the bioengineer has designated. The luciferase enzyme can be purified or partially purified from a microbial culture. Additional oxydising cofactors can be used, such as a flavin cofactor, a flavin mononucleotide (FMN), and/or a nicotinamide adenine dinucleotide (NAD). The mixture comprising the luciferase enzyme can further comprise at least one excipient such as, for example a sucrose, a trehalose, a mannitol, a polyvinylpyrrolidone (PVP), a polysorbate, a dextrose, a glycine and a combination thereof. In various embodiments, the at least one excipient may stabilize the luciferase enzyme(s) and optionally the at least one cofactor. In various embodiments, at least one excipient may also be added to the luciferin granulates. For example, during manufacture of the dried unreactive formulation by a freeze-drying process, at least one excipient may be added to the luciferin(s) to act as a cryoprotectant. In various embodiments, the at least one excipient may be added to the mixture of luciferase enzyme(s) and optionally the at least one cofactor before or after the mixture has been dried.

In another embodiment, the bioluminescent composition further comprises at least one adhesive, surfactant, thickener, or dispersant. The bioluminescent composition can be processed into a paste, a paint, or an ink formulation, by mixing the composition powder with chemicals that include glue/adhesive polymers such as polyurethane, polyimide, epoxy, acrylic, polyamide, polyester and protein, surfactants, thickeners, dispersants etc. The bioluminescent composition can then temporarily or permanently bind to a target material surface such as textiles, sports equipment or garments, skin, or hair though different processes such as coating, painting, laminating, printing etc. In another embodiment, the bioluminescent composition can be encapsuled and incorporated into a solid material form by mixing the composition powder or the encapsulated composition powder with polymers and other chemical agents such as dispersants to form a masterbatch, and then spinning, extruding, molding, casting, thermoforming or foaming the masterbatch into different forms such as a fiber, mat, film, foam or other solid forms.

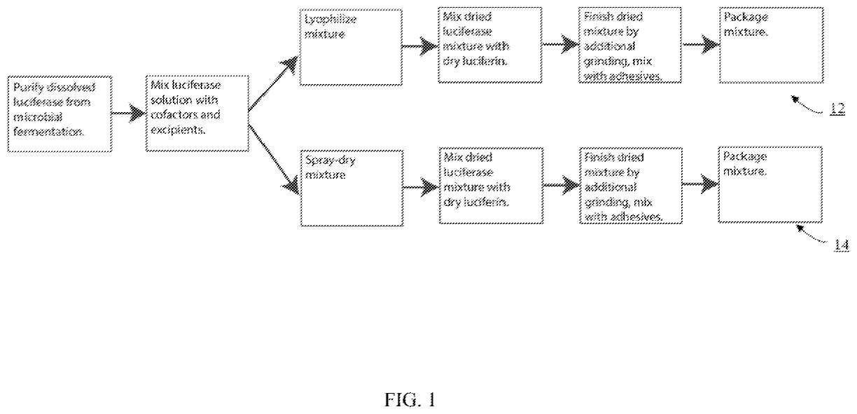

FIG. 1 schematically illustrates one method for manufacturing a moisture induced bioluminescent composition. The method comprises first a step of purifying or partially purifying a dissolved luciferase from a microbial fermentation culture and then mixing the luciferase solution with at least one cofactor and/or at least one excipient to form a luciferase mixture. The luciferase solution mixture is dried using a lyophilization technique (12) or a spray-drying technique (14) so that a dried luciferase mixture is obtained. The dried luciferase mixture is then mixed with a dried luciferin and the mixture is further mixed and ground to the desired granulate/powder size to form a dried unreactive bioluminescent composition. The dried unreactive bioluminescent composition is then packaged. For example, the dried unreactive bioluminescent composition can be packaged as an aerosol, an ink, a paint, a paste, a film, a capsule or in any other suitable packaging. Then such unreactive bioluminescent composition can be applied to surfaces, such as apparel, one or more body parts, hair by spraying, brushing, painting, coating, laminating or printing (such as, for example, 3D printing).

The lyophilization technique is typically performed in 3 stages: freezing, primary drying, and secondary drying. The freezing step includes freezing the luciferase mixture at a predetermined rate of freezing. The rate of freezing can vary such as, for example, at a slow rate of few degrees per hour (e.g., 1-5° C./hour) to very rapidly (1-5° C./s). In some embodiments, the rate of freezing can be at moderate speed (e.g., 1-5° C./min). The final freezing temperature can vary from 0° C. to −80° C. During the primary drying the bulk of water is removed by using low pressures to sublimate the matrix and it can be performed at various temperatures from 0° C. to −80° C. and at various pressures. For example, the primary drying can be performed at pressures just below atmospheric pressure (e.g. 750 Torr) to near vacuum (e.g. 40 mTorr). More vacuum can speed up the drying but may cause fracturing of the materials. Slower drying is gentler but slows the overall process. The time duration for the primary drying will vary depending on the volume of the formulation to be dried, the water content, freezing temperature, and pressure. Typically, it will last 8-12 hours.

During the secondary drying of the lyophilization technique, a remaining portion of the liquid is removed from the luciferase mixture. Following primary drying, the temperature is increased to near ambient (e.g. 1 to 30° C.) and low pressures of about 750 Torr-40 mTorr are maintained to draw off any remaining water.

The spray technique includes dispersing the luciferase mixture through a spray nozzle into a hot gas. The hot gas can flow in the same direction or counter flow to the dispersed luciferase mixture. Depending on a nozzle size and shape of the nozzle, a flow rate of the luciferase mixture can be 1-50 ml/min, a flow rate of the hot gas can be 100-1600 l/h, a temperature can be −20° C.-100° C. and a pressure can be 750 Torr-40 mTorr.

The bioluminescent composition can be activated by water, sweat, rain, fog or moisture that is generated by the human body or the environment. External water resources can be applied for the activation of the bioluminescent effect when less than enough water resource is available on the surface. The bioluminescent composition may also be micro-encapsulated into vesicles. Such vesicles may be dry on the interior but have container walls that become porous in water. In such a case, breaching of the vesicle and hydration of the composition may lead to a brighter “flash” of light.

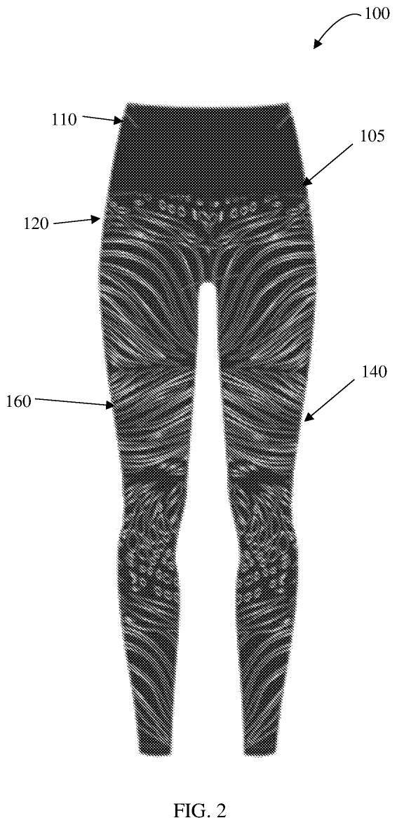

FIG. 2 illustrates an example of an article of apparel, such as, for example, a pair of tights 100 that comprises a body 105 made of a fabric with an outer face surface and an inner surface. The body 105 may include a waistband 110 forming a waist opening, an upper portion 120 configured to cover a front pelvic area and a back bottom area of a wearer, and two leg portions 140 configured to cover at least a portion of the legs of the wearer. A bioluminescent composition 160 is applied to at least a portion of the article of apparel 100 by spraying, brushing, painting coating, laminating or printing. In various embodiments, the bioluminescent composition 160 may form a pattern or design. The applied bioluminescent composition 160 is dormant or inactive on the outer face surface of the body 105 and is activated by water, sweat, rain, fog or moisture that is generated by the body of the wearer or the environment of the wearer, resulting in the article of apparel emitting light (luminescence). For example, the sweat generated by the wearer during athletic activity induces the bioluminescent composition 160 to produce luminescence, thereby providing different patterns or changes to the appearance of the pair of tights 100. As a further example, rain induces the bioluminescent composition 160 to produce luminescence, thereby increasing the visibility of the wearer for safety benefits. The article of apparel may be any other garment including, but not limited to, a long sleeve shirt, a short sleeve shirt, a tank top, a jacket, a poncho, a pair of pants, a pair of shorts, a dress, a skirt, a skort, footwear (such as, for example, a pair of shoes or a pair of socks), a sweater, a hat, a scarf, a bra or any other article of apparel.

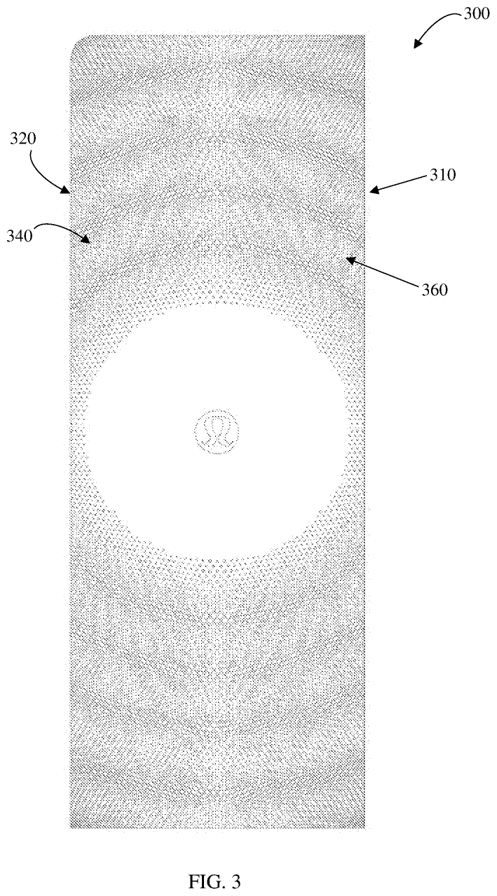

In various embodiments, the bioluminescent composition of the present invention may be applied to other surfaces such as exercise equipment, such as, for example, an exercise mat 300 as shown in FIG. 3. The exercise mat 300 has a body 310 with a first surface 320 and a second surface 340. For example, the first surface 320 may be on a surface such as a floor, and the second surface 340 is facing away from the floor. A bioluminescent composition 360 is applied to at least one of the first surface 320 or the second surface 340, or to both. For example, the bioluminescent composition 360 may be applied to the second surface 340 to alert a user that the exercise equipment, for example the mat 300, is wet. When the second surface 340 of the mat 300 is wet due to sweating or water, the bioluminescent composition 360 will be activated to produce luminescence that visually alerts the user to the wet surface. Persons of ordinary skill in the art would understand that the bioluminescent composition may be applied to any other surface or equipment including, but not limited to, a floor, a block, a seat, a handle, a handlebar, a step, or a stool, without departing from the scope of the invention.

The response of the bioluminescent composition can have various desired effects. This may include an increase in the garment's luminescence as the wearer's exercise intensity increases and sweat rehydrates the garment. The resulting luminescence may be bright enough to provide a safety benefit at night or it can be used as a sensor for a wearable device or augmented reality integration. The luminescent photons may be beyond those of visible light and/or so subtle as to require instrumentation to read, which may provide applications for tracing or augmented reality visualization. The color of the luminescence may change as the temperature of the wearer's skin increases or decreases; a similar effect may be achieved by a change in the wearer's skin moisture level, sweat pH, sweat salt content, lactate content, or other molecular changes in sweat composition. The wearer may also “pre-wet” the finished garment to activate the luminescence prior to exercise. Some formulations may preclude wetting the material and may instead be sufficiently active in dried state and activate only upon exposure to oxygen.

While particular elements, embodiments and applications of the present disclosure have been shown and described, it will be understood that the scope of the disclosure is not limited thereto, since modifications can be made by those skilled in the art without departing from the scope of the present disclosure, particularly in light of the foregoing teachings. Thus, for example, in any method or process disclosed herein, the acts or operations making up the method/process may be performed in any suitable sequence and are not necessarily limited to any particular disclosed sequence. Elements and components can be configured or arranged differently, combined, and/or eliminated in various embodiments. The various features and processes described above may be used independently of one another, or may be combined in various ways. All possible combinations and subcombinations are intended to fall within the scope of this disclosure. Reference throughout this disclosure to “some embodiments,” “an embodiment,” or the like, means that a particular feature, structure, step, process, or characteristic described in connection with the embodiment is included in at least one embodiment. Thus, appearances of the phrases “in some embodiments,” “in an embodiment,” or the like, throughout this disclosure are not necessarily all referring to the same embodiment and may refer to one or more of the same or different embodiments. Indeed, the novel composites, textiles and methods described herein may be embodied in a variety of other forms; furthermore, various omissions, additions, substitutions, equivalents, rearrangements, and changes in the form of the embodiments described herein may be made without departing from the disclosure described herein.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without operator input or prompting, whether these features, elements and/or steps are included or are to be performed in any particular embodiment. No single feature or group of features is required for or indispensable to any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The example calculations, simulations, results, graphs, values, and parameters of the embodiments described herein are intended to illustrate and not to limit the disclosed embodiments. Other embodiments can be configured and/or operated differently than the illustrative examples described herein.

1. A moisture induced bioluminescent composition comprising:
luciferin granulates; and
a mixture of one or more dried luciferase enzymes and one or both of at least one cofactor and at least one excipient,
wherein the luciferin granulates are mixed with the dried luciferase mixture forming an unreactive composition, the unreactive composition configured to produce a luminescence when exposed to moisture.

2. The moisture induced bioluminescent composition of claim 1, wherein the luciferin granulates comprise a D-luciferin, a coelenterazine, and a vargulin/cypridinaluciferin.

3. The moisture induced bioluminescent composition of claim 1, wherein the luciferase enzyme is purified or partially purified from a microbial culture.

4. The moisture induced bioluminescent composition of claim 1, wherein the at least one excipient is selected from the group consisting of a sucrose, a trehalose, a mannitol, a polyvinylpyrrolidone (PVP), a polysorbate, a dextrose, a glycine and a combination thereof.

5. The moisture induced bioluminescent composition of claim 1, wherein the at least one cofactor is a flavin cofactor, a flavin mononucleotide (FMN) and/or a nicotinamide adenine dinucleotide.

6. The moisture induced bioluminescent composition of claim 1, further comprising at least one of an adhesive, a surfactant, a thickener, or a dispersant.

7. The moisture induced bioluminescent composition of claim 6, wherein the at least one adhesive is selected from the group consisting of a polyurethane, a polyimide, an epoxy, an acrylic, a polyamide, a polyester and a protein.

8. A method of manufacturing a moisture induced bioluminescent composition comprising:
purifying a dissolved luciferase from a microbial fermentation culture to form a luciferase solution;
mixing the luciferase solution with at least one cofactor and/or at least one excipient forming a luciferase mixture;
drying the luciferase mixture using a lyophilization technique or a spray-drying technique to form a dried luciferase mixture;
mixing the dried luciferase mixture with a dried luciferin;
grinding the mixture of dried luciferase mixture and the dried luciferin to form a dried unreactive bioluminescent composition; and
packaging the dried unreactive bioluminescent composition to form the moisture induced bioluminescent composition.

9. The method of claim 8, further comprising mixing at least one of an adhesive, a surfactant, a thickener, or a dispersant with the dried unreactive bioluminescent composition.

10. The method of claim 8, wherein the lyophilization technique comprises freezing the luciferase mixture at a temperature of 0° C. to −80° C. at a predetermined rate of freezing to form a frozen luciferase mixture, primary drying the frozen luciferase mixture at a pressure of 750 Torr-40 mTorr for 8-12 hours and maintaining the temperature of 0° C. to −80° C. to sublimate a frozen liquid away from the frozen luciferase mixture; and secondary drying the frozen luciferase mixture at temperature of 1-30° C. while maintaining the pressure of 750 Torr-40 mTorr to remove any remaining liquid from the luciferase mixture to form the dried luciferase mixture.

11. The method of claim 8, wherein the spray technique comprises dispersing the luciferase mixture through a spray nozzle into a hot drying gas, wherein a flow rate of the luciferase mixture is 1-50 ml/min, a flow rate of the hot gas is 100-1600 l/h, a temperature is −20° C.-100° C., and a pressure is 750 Torr-40 mTorr.

12. The method of claim 8, wherein the unreactive bioluminescent composition is packaged as an aerosol or a paste.

13. The method of claim 8, wherein the unreactive bioluminescent composition is packaged in a capsule.

14. An article of apparel comprising:
a body configured to fit and cover at least a portion of a body of a wearer, the body having an outer surface facing away from the body of the wearer; and
a moisture induced bioluminescent composition of claim 1 applied to at least a part of the outer surface of the body.

15. The article of apparel of claim 14, wherein the moisture induced bioluminescent composition is coated, sprayed, printed, painted or brushed to the part of the outer surface of the body.
